(19) United States
(12) Patent Application Publication
(10) Pub. No.: US 2014/0268651 A1
(43) Pub. Date: Sep. 18, 2014

(54) FRAGRANCE WARMER AND LIGHT STRING ASSEMBLY HAVING THE SAME

(71) Applicants: Richard Russel Mumma, Southlake, TX (US); Yale Spitzer, Chicago, IL (US)

(72) Inventors: Richard Russel Mumma, Southlake, TX (US); Yale Spitzer, Chicago, IL (US)

(73) Assignee: INTERNATIONAL DEVELOPMENT LLC, Roanoke, TX (US)

(21) Appl. No.: 14/212,140

(22) Filed: Mar. 14, 2014

Related U.S. Application Data
(60) Provisional application No. 61/783,748, filed on Mar. 14, 2013.

(51) Int. Cl. A61L 9/03; F21V 33/00

(52) U.S. Cl. CPC A61L 9/03; F21V 33/0092
USPC 362/96; 392/386

(57) ABSTRACT

A fragrance-producing light string assembly can be achieved using either a fragrance warmer configured to connect to a conventional light string assembly or a light string assembly having an integrated fragrance warmer. The fragrance warmer is configured to apply heat to a removable and replaceable fragrance pellet that is formed from a polymeric material that is impregnated with a vaporizing agent and configured to emit the vaporizing agent when heated. The fragrance warmer includes a heating element and a heating plate arranged to apply heat to the fragrance pellet using electrical power from the light string assembly.

This application claims the benefit of U.S. Provisional Application No. 61/783,748, filed 14 Mar. 2013, which is hereby incorporated herein by reference.

### TECHNICAL FIELD

The present disclosure relates to decorative lights, and more particularly to strings of decorative lights. The present disclosure further relates to fragrance-emitting apparatus.

### SUMMARY

According to some aspects of the present disclosure, a fragrance warmer comprises a base portion and a warming portion. The base portion is configured to connect physically and electrically to a light socket of a string light assembly. The warming portion comprises a fragrance holder configured to hold a fragrance pellet, a heating plate arranged to apply heat to the fragrance pellet, and a heating element arranged to receive electrical power from the string light assembly and transfer heat to the heating plate.

In some embodiments, the warming portion can be directly supported by the base portion. In other embodiments, an electrical cord can be connected between the base portion and the warming portion.

The fragrance warmer can include the fragrance pellet. The fragrance pellet can be formed of a polymeric material that is impregnated with a vaporizing agent and configured to emit the vaporizing agent when heated by the heating element.

According to another aspect of the present disclosure, a string light assembly can comprise an electrical cord electrically connecting a series of light sockets and at least one fragrance socket. The fragrance socket can be configured to hold a fragrance pellet. The fragrance socket can include a heating plate arranged to apply heat to the fragrance pellet, and a heating element arranged to receive electrical power from the electrical cord and transfer heat to the heating plate. The fragrance warmer can include a fragrance pellet formed of a polymeric material that is impregnated with a vaporizing agent and configured to emit the vaporizing agent when heated by the heating element.

### BRIEF DESCRIPTION OF THE DRAWINGS

Features, aspects, and embodiments of the present disclosure are described in conjunction with the attached drawings, in which:

FIG. 1 shows a conventional string light assembly;

FIG. 2 shows a decorative light being removed from the string light assembly shown in FIG. 1;

FIG. 3 shows partially-sectioned side view of a fragrance warmer according to the present disclosure;

FIGS. 4 and 5 show the fragrance warmer shown in FIG. 3 being attached to the string light assembly shown in FIG. 1;

FIG. 6 shows an alternative string light assembly according to the present disclosure;

FIG. 7 shows an enlarged, partially sectioned view of a fragrance socket of the string light assembly shown in FIG. 6;

FIG. 8 shows a side view of the fragrance pellet used with the fragrance socket shown in FIGS. 6 and 7; and

FIG. 9 shows an alternative embodiment of a fragrance warmer that can be used with a conventional string light assembly such as the string light assembly shown in FIGS. 1 and 2.

### DETAILED DESCRIPTION

FIG. 1 shows a conventional light string assembly 100 such as those known to have various lengths and numbers of removable decorative lights 102. The light string 100 shown in FIG. 1 specifically includes decorative lights 102a, 102b, 102c, and 102d, which are electrically and physically interconnected by an electrical cord 104. The electrical cord 104 includes a plurality of light sockets 106a, 106b, 106c, and 106d, which are spaced at various locations along the length of electrical cord 104 between an electrical plug 108 at one end and an electrical receptacle 110 at an opposite end.

The decorative lights 102a-102d are individually removable by sliding them from their respective light sockets 106a-106d. For example, FIG. 2 shows the decorative light 102c being removed by sliding it from its light socket 106c. The decorative light 102c includes a slide-in style base portion 112 for mating with the light socket 106c. Alternatively, the decorative lights 102a-102d and their respective light sockets 106a-106d can be provided with other types of connections known in the art, for example threaded or bayonet types of connections. The base portion 112 includes exposed wires 114a and 114b that receive electrical power from the socket 106c and transfer the electrical power to the decorative light 102c for illumination.

Turning next to FIG. 3, a fragrance warmer 200 is shown that can be used in place of the decorative light 102c, as shown in FIGS. 5 and 6.

FIG. 3 shows a partially-sectioned side view of the fragrance warmer 200. The fragrance warmer 200 includes a base portion 202 and a warming portion 204. In use, one or more of the fragrance warmers 200 can replace one or more decorative lights 102 in a light string assembly 100 so that the light string 100 can be used to emit a fragrance in addition to providing decorative lighting. Thus, the base portion 202 of the fragrance warmer 200 is preferably constructed to substantially match the base portion 112 of the decorative light 102c that the fragrance warmer 200 is intended to replace. As such, the base portion 202 includes exposed wires 206a and 206b that receive electrical power from the socket 106c and transfer the electrical power to the warming portion 204 for generating heat.

The warming portion 204 includes a fragrance holder 208, a heating plate 210, and a heating element 212. The heating element 212 is connected to wires 214a and 214b, which are electrically connected to wires 206a and 206b, respectively. The fragrance holder 208 is preferably formed of a material that resists transfer of heat, such as a plastic or rubber material. The heating plate 210 is preferably formed of a material that efficiently transfers heat, such as a metal, for example aluminum. The heating element 212 is an element that can convert electricity into heat, for example a positive thermal coefficient (PTC) ceramic.

In use, the heating element 212 can receive electrical power from the electrical cord 104 via wires 206a, 206b, 214a, and 214b. The received electrical power causes the heating element 212 to increase in temperature and transfer heat to the heating plate 210. The heating element 212 and the heating plate 210 eventually reach a maximum operating temperature, which can be set, for example, based on the composition of the heating element 212 and/or the amount of electrical power being provided to the heating element 212. The heating plate 210 can then be used to transfer heat to a fragrant material, such as fragrance pellet 250 shown in FIGS. 4 and 5, so that the fragrant material will emit its fragrance. Thus, the maximum operating temperature of the heating plate 210 can be selected based on the fragrant material being used.

Referring to FIGS. 4 and 5, a fragrance pellet 250 can be inserted into, and frictionally retained within, the fragrance holder 208. When inserted as shown in FIG. 5, the fragrance pellet 250 makes contact with the upper surface of the heating plate 210 so that, during operation, the heating plate 210 can apply heat to the fragrance pellet 250. The fragrance pellet 250 is the source of the fragrance produced by the fragrance warmer 200 and is replaceable by a user. The fragrance pellet 250 of the present embodiment is cylindrical in shape, having a circular cross-section. However, other shapes can be used. The fragrance pellet 250 of the present embodiment is solid. However, in some embodiments, the fragrance pellet 250 can be perforated such that it includes one or more venting holes extending therethrough.

The fragrance pellet 250 can be at least partially composed of a polymeric material, for example ethylene vinyl acetate (EVA), which is a known copolymer of ethylene and vinyl acetate that is flexible and can remain in a substantially solid state when heated by the heating plate 210. The fragrance pellet 250 can be configured so as to not substantially deform at temperatures of less than two hundred degrees Fahrenheit, although some shrinkage of the disk can occur over time as the vaporizing agents are dispersed into the air.

As those with skill in the art will appreciate, the properties of EVA allow it to be impregnated, embedded, or absorbed in or infused with (collectively referred to herein by use of the term “impregnate” or variations thereof) various vaporizing agents such as, for example, a fragrance, permitting it to be used as a fragrance reservoir in the device. It should be noted that for the purposes of this specification and the claims recited herein, the term “vaporizing agent” should be construed to mean any substance that may exist in a substantially solid and or liquid state but that is also capable of existing in a substantially vaporized and/or aerosolized state when heated.

Methods for making and using EVA and other polymeric materials and impregnating them with vaporizing agents such as fragrances, which can include odor-neutralizing substances, insecticides and substances having medicinal properties that are known in the art. EVA material of the fragrance pellet 250 can have a molecular weight in the range of 10,000 Daltons to 100,000 Daltons. Vaporizing agents may be impregnated into the EVA fragrance pellet 250 at weight percents varying from 10 to 90%, from 20 to 80%, from 30 to 70%, from 30 to 60%, and from 30 to 50%. For example, in some embodiments, the fragrance disk can be composed of about 75% EVA by weight and about 25% of fragranced vaporizing agent by weight. It should be noted however, that alternative embodiments of the fragrance pellet 250 may include other combinations by weight of the polymeric material and the vaporizing agent.

Alternative embodiments of the fragrance pellet 250 may be composed of other polymeric materials including, but not limited to, polyethylene (high or low density), polypropylene, polyvinyl chloride, polystyrene, polycarbonate, acrylonitrile butadiene styrene (ABS), PEBAX and polymethylpentene, ethyl vinyl alcohol, polystyrene, acrylic polymers, polycarbonates, polyurethanes, and nylons.

Possible fragrances to be impregnated in the fragrance pellet 250 may be selected from the non-exhaustive list of fragrances including musk oil, civet, castreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, cauout oil, salvia oil, clove oil, chamomile oil, costus oil, labdanum oil, broom extract, carrot seed extract, jasmine extract, minmosa extract, narcissus extract, olibanum extract, rose extract, acetophenonene, dimethylinadane derivatives, naphthaline derivatives, allyl caprate, alpha-amylcinnamic aldehyde, anethole, anisaldehyde, benzyl acetate, benzyl alcohol, benzyl propionate, borneol, cinnmayl acetate, cinnamyl alcohol, citral citronellal, cumin aldehyde, cyclamen aldehyde, decanol, ethyl butyrate, ethyl caprate, ethyl cinnamate, ethyl vanillin, eugenol, geraniol, exenol, alpha-hexylcinnamic aldehyde, hydroxycitrolnellal, indole, iso-amyl acetate, iso amyl isovaleratek iso-eugenol, linalol, linalyl acetate, p-methylacetophenone, methyl anthranilate, methyl dihydroasmonate, methyl eugenol, methyl-beta-naphthol ketone, methylphenhlcarbinyl acetate, musk ketol, mustk xylol, 2,5,6nanodinol, gannna-nanolactone, phenylacetoaldehydrodimethyl acetate, beta-phenylethyl alcohol, 3,3,5-trimethylcyclohexanol, gamma-undecalactone, undecenal, vanillin, and mixtures thereof. The foregoing hindered amines may be added to the fragranced vaporizing agent at weights of between 0.1% and 2.0% by weight of the EVA, preferably at 0.7% by weight. Those of skill in the art will recognize that in constructing the fragrance disk, it is also desirable to mix the fragrance with hindered amines such as for example, 1-(2-hydroxy-2-methylpropoxy)-4-octadecanoyloxy-2,2,6,6-tetramethylpiperi-dine; 1-(2-hydroxy-2-methylpropoxy)-4-hydroxy-2,2,6,6-tetramethylpiperidin-e; and bis(1-octyloxy-2,2,6,6-tetramethylpiperidin-4-yl)sebacate. Further, various antioxidants such as tertiary butylhydroquinone, butylated hydroxyanisole, phenol bisphosphite, and butylated hydroxytoluene are preferably added to the fragranced vaporizing agent at amounts of between 0.015% and 2.5% by weight of the EVA or other polymer, and preferably between 0.2% and 0.5% by weight.

It is contemplated that in alternative embodiments the EVA material used in the fragrance pellet 250 may also be impregnated with other vaporizing agents such as an insecticide. The insecticide may be selected from the non-exhaustive list of substances including citronella, pyrethrum, methyl bromide, aluminum phosphide, and magnesium phosphide. Other alternative embodiments may also utilize a pellet 250 impregnated with vaporizing agents having medicinal properties. Such vaporizing agents having medicinal properties may be selected from the non-exhaustive list of preparations including eucalyptus oil, menthol, camphor, thymol, turpentine oil, 1-desoxyephedrine, and bornyl acetate. In other alternative embodiments, the foregoing substances may be impregnated in the pellet 250 in combination with one or more other fragrances or alone. In other words, it is contemplated that alternative embodiments of the pellet 250 as disclosed and claimed herein, may or may not be impregnated with fragrances, which can include one or more vaporizing agents not chosen primarily for the production of a pleasing aroma.

The beneficial properties of the EVA material used in the pellet 250 provide for the distribution of heat necessary to permit an approximately uniform heating effect throughout said pellet 250. When properly heated, the configuration of the pellet 250 allows warm air via convection to lift fragrant vaporizing agents from the pellet 250 into the ambient air. To facilitate this aspect of dispersal, a plurality of smaller (smaller than the central aperture) holes can be placed throughout the body of said pellet 250 to accommodate the effect of convection.

During operation of the present embodiment of the fragrance warmer 200, the upper surface of the heating plate 210 can be heated by the heating element 212 from room temperature to a temperature in a range of approximately 100 degrees Fahrenheit to approximately 150 degrees Fahrenheit. For example, in some embodiments, the upper surface of the heating plate 210 can heat from room temperature to a temperature in a range of approximately 110 degrees Fahrenheit to approximately 135 degrees Fahrenheit. In some such embodiments, the upper surface of the heating plate 210 can heat from room temperature to a temperature in a range of approximately 118 degrees Fahrenheit to approximately 126 degrees Fahrenheit, such as, for example, approximately 122 degrees Fahrenheit.

Referring next to FIGS. 6-8, an alternative embodiment is disclosed wherein the string light assembly is a fragrance-producing string light assembly that includes one or more sockets having integrated heating elements for holding and warming fragrance-emitting elements.

FIG. 6 shows a fragrance-producing light string assembly 300. The string light assembly 300 can have various lengths and numbers of removable decorative lights 102. The light string 300 shown in FIG. 6 specifically includes decorative lights 102a, 102b, and 102c, which are electrically and physically interconnected by an electrical cord 104. The electrical cord 104 includes a plurality of light sockets 106a, 106b, and 106c, as well as a fragrance socket 304, which are spaced at various locations along the length of electrical cord 104 between an electrical plug 108 at one end and an electrical receptacle 110 at an opposite end.

The decorative lights 102a-102c are individually removable by sliding them from their respective light sockets 106a-106c, for example in a manner as shown in FIG. 2. Alternatively, the decorative lights 102a-102c and their respective light sockets 106a-106c can be provided with other types of connections known in the art, for example threaded or bayonet types of connections.

A fragrance pellet 302 is individually removable from the fragrance socket 304. FIG. 8 shows a side view of the fragrance pellet 302. The fragrance pellet 302 can be the same as fragrance pellet 250, except that the upper portion (the portion of the fragrance pellet 302 that is exposed when the fragrance pellet 302 is in the fragrance socket 304) of the fragrance pellet 302 has been formed to at least somewhat match the shape of the decorative lights 102.

FIG. 7 shows an enlarged, partially sectioned view of the fragrance socket 304 with the fragrance pellet 302 inserted therein. The fragrance socket 304 includes an outer shell 308, a heating plate 310 disposed within the outer shell 308, and a heating element 312 disposed adjacent to the heating plate 310. The heating element 312 is connected to wires 316a and 316b, which are electrically connected to electrical cord 104. The outer shell 308 is preferably formed of a material that resists transfer of heat, such as a plastic or rubber material. The heating plate 310 is preferably formed of a material that efficiently transfers heat, such as a metal, for example aluminum. Also, the present embodiment clarifies that the term “plate” is not necessarily limited to flat-shaped objects, as the heating plate 310 is shaped somewhat like a cup. The heating element 312 is an element that can convert electricity into heat, for example a positive thermal coefficient (PTC) ceramic.

In use, the heating element 312 can receive electrical power from the electrical cord 104 via wires 316a and 316b. The received electrical power causes the heating element 312 to increase in temperature and transfer heat to the heating plate 310. The heating element 312 and the heating plate 310 eventually reach a maximum operating temperature, which can be set, for example, based on the composition of the heating element 312 and/or the amount of electrical power being provided to the heating element 312. The heating plate 310 can then be used to transfer heat to a fragrant material, such as fragrance pellet 302, so that the fragrant material will emit its fragrance. Thus, the maximum operating temperature of the heating plate 310 can be selected based on the fragrant material being used.

The fragrance pellet 302 can be inserted into, and frictionally retained within, the fragrance socket 304. When inserted as shown in FIGS. 6 and 7, the fragrance pellet 302 makes contact with the heating plate 310 so that, during operation, the heating plate 310 can apply heat to the fragrance pellet 302. The fragrance pellet 302 is the source of the fragrance produced by the fragrance-producing string light assembly 300 and is replaceable by a user. While the fragrance-producing string light assembly 300 is shown with a single fragrance socket 304, alternative embodiments of the fragrance-producing string light assembly 300 can include any number of fragrance sockets 304. The fragrance pellet 302 of the present embodiment is similar to a decorative light in shape, having a cylindrical lower portion and a light-shaped upper portion as shown in FIG. 8. However, other shapes can be used. The fragrance pellet 302 of the present embodiment is solid. However, in some embodiments, the fragrance pellet 302 can be perforated such that it includes one or more venting holes extending therethrough. The description of the fragrance pellet 250, including the fragrances and composition thereof, applies equally to fragrance pellet 302.

FIG. 9 shows another alternative embodiment where a fragrance pellet holder is provided with a cord and a connector that can be connected to a light socket of a string light assembly.

FIG. 9 shows a light string assembly 100, which can be a conventional string light assembly such as those known to have various lengths and numbers of removable decorative lights 102. The light string 100 shown in FIG. 9 specifically includes decorative lights 102a, 102b, and 102d, which are electrically and physically interconnected by an electrical cord 104. The electrical cord 104 includes a plurality of light sockets 106a, 106b, 106c, and 106d, which are spaced at various locations along the length of electrical cord 104 between an electrical plug 108 at one end and an electrical receptacle 110 at an opposite end.

As discussed above in connection with FIG. 2, the decorative lights 102a, 102b, and 102d are individually removable by sliding them from their respective light sockets 106a, 106b, and 106d. For example, FIG. 2 shows a decorative light 102c being removed by sliding it from the light socket 106c. The decorative light 102c includes a slide-in style base portion 112 for mating with the light socket 106c. Alternatively, the decorative lights 102a-102d and their respective light sockets 106a-106d can be provided with other types of connections known in the art, for example threaded or bayonet types of connections. The base portion 112 includes exposed wires 114a and 114b that receive electrical power from the socket 106c and transfer the electrical power to the decorative light 102c for illumination.

Referring again to FIG. 9, a fragrance warmer 400 is shown that can be used in place of the decorative light 102c.

The fragrance warmer 400 includes a warming portion 202, which can stand alone or be supported within a housing, such as a decorative housing 404 of any desired shape. In use, one or more of the fragrance warmers 400 can replace one or more decorative lights 102 in a light string assembly 100 so that the light string 100 can be used to emit a fragrance in addition to providing decorative lighting. Thus, the fragrance warmer 400 includes an electrical cord 414 connected to the warming portion 402, and further includes a socket connector 416 connected to the electrical cord 414. The socket connector 416 is preferably constructed to substantially match the base portion 112 of the decorative light 102c (shown in FIG. 2) that the fragrance warmer 400 is intended to replace. As such, the socket connector 416 can include exposed wires that receive electrical power from the socket 106c and transfer the electrical power to the warming portion 402 for generating heat.

The warming portion 402 includes a fragrance holder 408, a heating plate 410, and a heating element 412. The heating element 412 is connected to electrical cord 414, which are electrically connected to exposed wires of the socket connector 416. The fragrance holder 408 is preferably formed of a material that resists transfer of heat, such as a plastic or rubber material. The heating plate 410 is preferably formed of a material that efficiently transfers heat, such as a metal, for example aluminum. The heating element 412 is an element that can convert electricity into heat, for example a positive thermal coefficient (PTC) ceramic.

In use, the heating element 412 can receive electrical power from the electrical cord 104 via the electrical cord 414. The received electrical power causes the heating element 412 to increase in temperature and transfer heat to the heating plate 410. The heating element 412 and the heating plate 410 eventually reach a maximum operating temperature, which can be set, for example, based on the composition of the heating element 412 and/or the amount of electrical power being provided to the heating element 412. The heating plate 410 can then be used to transfer heat to a fragrant material, such as fragrance pellet 406, so that the fragrant material will emit its fragrance. Thus, the maximum operating temperature of the heating plate 210 can be selected based on the fragrant material being used. The fragrance pellet 406 can be identical to the fragrance pellet 250 shown in FIGS. 4 and 5 or the fragrance pellet 302 shown in FIG. 8. Thus, the description of the fragrance pellet 250 applies equally to the fragrance pellet 406.

The fragrance pellet 406 can be inserted into, and frictionally retained within, the fragrance holder 408. When inserted as shown in FIG. 9, the fragrance pellet 406 makes contact with the upper surface of the heating plate 410 so that, during operation, the heating plate 410 can apply heat to the fragrance pellet 406. The fragrance pellet 406 is the source of the fragrance produced by the fragrance warmer 400 and is replaceable by a user. The fragrance pellet 406 of the present embodiment is cylindrical in shape, having a circular cross-section. However, other shapes can be used. The fragrance pellet 406 of the present embodiment is solid. However, in some embodiments, the fragrance pellet 406 can be perforated such that it includes one or more venting holes extending therethrough.

While the fragrance pellets 250, 302, and 406 have been described herein as being frictionally retained within respective holders, alternatively a great variety of retaining means can be used to secure the fragrance pellets in place, such as barbs, clips, caps, snaps, or other known connection means.

The embodiments described herein provide for string light assemblies that can either be converted to emit fragrance or can be manufactured to emit fragrance using replaceable fragrance pellets. Such string light assemblies can be useful for adding fragrance to a Christmas tree or a room where decorative string lights are hung. Also, such string light assemblies can be used to illuminate an outdoor area, and fragrance pellets having insect-repelling properties can be used in the string light assembly to help keep the illuminated area free of insects.

While various embodiments in accordance with the disclosed principles have been described above, it should be understood that they have been presented by way of example only, and are not limiting. Thus, the breadth and scope of the invention(s) should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

What is claimed is:

1. A fragrance warmer, comprising:
a base portion configured to connect physically and electrically to a light socket of a string light assembly; and
a warming portion comprising:
a fragrance holder configured to hold a fragrance pellet;
a heating plate arranged to apply heat to the fragrance pellet; and
a heating element arranged to receive electrical power from the string light assembly and transfer heat to the heating plate.

2. The fragrance warmer of claim 1, wherein the warming portion is directly supported by the base portion.

3. The fragrance warmer of claim 1, further comprising an electrical cord connected between the base portion and the warming portion.

4. The fragrance warmer of claim 1, further comprising the fragrance pellet, wherein the fragrance pellet is formed of a polymeric material that is impregnated with a vaporizing agent and configured to emit the vaporizing agent when heated by the heating element.

5. A string light assembly, comprising:
an electrical cord electrically connecting a series of light sockets and at least one fragrance socket, the fragrance socket being configured to hold a fragrance pellet and comprising:
a heating plate arranged to apply heat to the fragrance pellet; and
a heating element arranged to receive electrical power from the electrical cord and transfer heat to the heating plate.

6. The fragrance warmer of claim 5, further comprising the fragrance pellet, wherein the fragrance pellet is formed of a polymeric material that is impregnated with a vaporizing agent and configured to emit the vaporizing agent when heated by the heating element.
